(19) United States
(12) Patent Application Publication
(10) Pub. No.: US 2005/0177093 A1
(43) Pub. Date: Aug. 11, 2005

(54) Joint / tissue inflammation therapy and monitoring device

(76) Inventors: Hart M. Barry, Ellicott City, MD (US); Henryk Malak, Ellicott City, MD (US)

Correspondence Address: ARMSTRONG, KRATZ, QUINTOS,;HANSON & BROOKS, LLP, Suite 220, 502 Washington Avenue, Towson, MD, 21204, US

(21) Appl. No.: 11/100,029

(22) Filed: Apr. 6, 2005

Related U.S. Application Data
(63) Continuation-in-part of application No. 10/366,267, filed on Feb. 14, 2003.
(60) Provisional application No. 60/361,161, filed on Mar. 4, 2002.

(51) Int. Cl. A61N 1/30

(52) U.S. Cl. 604/20; 607/86

(57) ABSTRACT

A device is provided, in direct skin contact, surrounding an injured area for the light and heat treatment, reduction of joint inflammation, edema and excitation of neural and muscular stimulation associated with human and mammal tissues. This therapeutic light and heat source includes multiple tiers or layers, e.g., three or four layers and a multiplicity of light emitting diodes (LED's) found in the ranges of 250 nm to 20,000 nm and fiber optic connections. A neoprene type material or other non-allergenic material will be used to set the LED's and fiber optics in layers consisting of contact with the skin to a few centimeters from the skin tissue. (Distance between the layers of LED's will vary from contact or near contact with devices to several millimeters of separation.) Each LED array will be independently controlled allowing for optimal modulation of light frequencies and wavelengths. Technology will be integrated allowing for biomedical feedback of tissue temperature, biochemical changes in tissue and other statistical information. A low voltage, portable power supply, will be integrated into the device as well as an analog/digital, input/output connection device. The design will be created for continuous wear, flexibility and comfort. The tiers or layers can be individually programmed to turn “on” or “off” as required by the physiologic condition to be treated. A device for light and heat treatment is contemplated using polarized film or a light guide accepting white light.

### RELATED APPLICATIONS

This application is related to U.S. Provisional Patent Application No. 60/361/161, entitled “Joint Inflammation Therapy and Monitoring Device”, filed Mar. 4, 2002, which is herein incorporated by reference, and is also a continuation-in-part of non-provisional patent application Ser. No. 10/366,267, filed Feb. 14, 2003.

### STATEMENT REGARDING FEDERALLY SPONSORED RESEARCH

There is NO claim for federal support in research or development of this project.

### FIELD OF INVENTION

The herein disclosed invention finds applicability in the field of phototherapy. Phototherapy or light-therapy is known to therapeutically influence various organs of the body to bring about healing.

### BACKGROUND OF THE INVENTION

Pathology of Inflammation

To reduce such pain and suffering as found in joint inflammation and tissue edema, which are associated with the conditions of muscular strains, muscular stress, arthritis, blunt trauma, surgical procedures . . . Common methods have been introduced to the public. This range from the use of external chemicals and ointments, cold and heat treatments to sophisticated physical therapy applied to the area of inflammation in question. The principle behind this action is to stimulate blood flow and circulation to the effected area Over the past several years, light technology and photosensitizing agents have been used to reduce edema to surrounding tissues during pre and post surgical procedures to sensitive areas such as the eyes.

When the body has been injured either by accident or through medical procedures there is going to be a period of time where inflammation and edema will set into the affected tissue area. This is a natural defense mechanism that is extremely valuable to the body, whether human or other mammal. To understand this problem in more detail, one must understand the five- (5) common signs of inflammation and the metabolic phases at which they occur. When in the process of reviewing or diagnosing a patient, the following signs are usually identified: (a) swelling (edema), (b) redness or discoloration, (c) radiant heat from the wounded site, (d) pain (tender to the touch) and (e) possible loss of motor or neurological functions due to the affected area. In addition, there are three primary metabolic phases through which inflammation progresses and they are usually identified as degenerative, vascular and healing. Of these three phases, the vascular and healing phases are of most concern to the design and application of the device of the herein disclosed invention. Hyper migration and activity of the “Inflammatory Cells” such as neutrophils, macrophages, lymphocytes, and monocytes, occur during changes in blood vessels identified as the vascular phase. From this hyperactivity, the capillary and postcapillary networks become flooded and expand causing hyperemia. Due to this proliferation of the capillaries, redness will present itself in the inflamed tissue. Normally, the blood temperatures in the dermal and epidermal layers of tissue are cooler due to external ambient temperatures. Increased blood flow to this damaged area of tissue increases the temperature to ranges that are similar to blood found in the heart or aorta. This effect is the heat or warm feeling that surrounds the wound or injured area.

The physiology of the human body to heal, is directly associated with the aforementioned cells (neutrophils and monocytes). These cells, as a family, are known as leukocytes. As they move along the blood vessel walls looking for fissures or gaps through which they can migrate, leukocytes begin to attack dying or dead cells. This begins a process of releasing a fluid that combines with a serous substance being extruded from the wall of the blood vessel. Later this process helps in the reduction of pathogenic microorganism to develop into the blood stream. Another cell, known as a platelet, begins the adhesion process to the walls of the damaged vessel. Fibrin fibers simultaneously appear forming a fine mesh and developing a “clot” which pulls the damaged edges of the wound together whether this is an internal tear or external laceration.

Some of the major conditions, syndromes, disease and associated disorders that will benefit from phototherapy are including, but not limited to: Ankylosing Spondylitis (SA), Avascular Necrosis (Osteonecrosis), Back Pain, Behcet's Disease, Bursitis and other Soft Tissue Diseases, Calcium Pyrophosphate Dihydrate, Crystal Deposition Disease (CPPD) (Pseudo Gout), Carpal Tunnel Syndrome, Connective Tissue-Related Diagnosis, Crohn's Disease, Dermatomyositis, Ehlers-Danlos Syndrome (EDS), Fibromyalgia, Giant Cell Arteritis and Polymyalgia Rheumatica Gout, Inflammatory Bowel Disease, Juvenile Arthritis and Related Conditions, Juvenile Dermatomyositis, Juvenile Non-Inflammatory Disorders, Juvenile Psoriatic Arthritis, Juvenile Rheumatoid Arthritis (JRA), Juvenile Scleroderma, Juvenile Spondyloarthropathy Syndromes, Juvenile Systemic Lupus Erythematosus (SLE), Juvenile Vasculitis, Lyme Disease, Mixed Connective Tissue Disease (MCTD), Marfan Syndrome, Myofascial Pain, Myositis (Polymyositis, Dermatomyositis) Osteoarthritis, Osteogenesis Imperfecta, Osteonecrosis, Osteoporosis, Paget's Disease, Polyarteritis Nodossa and Wegener's Granulomatosis, Polymyositis, Pseudooxanthoma Elasticum (PXE), Psoriatic Arthritis, Raynaud's Phenomenon, Reflex Sympathetic Dystrophy Syndrome, Reactive Arthritis (Reiter's Syndrome), Rheumatoid Arthritis, Sarcoidosis, Scleroderma, Sjogren's Syndrome, Still's Disease, Systemic Lupus Erythematosus (Lupus) and Tendonitis.

### BACKGROUND OF THE INVENTION

The phototherapy device of the herein disclosed invention, namely, the Joint/Tissue Inflammation Therapy and Monitoring Device of JITMon Device relate to the field of Photochemistry and Photobiology as it applies to inflammation, edema, muscular and neural stimulation of human and mammal skin tissues. As a design, this device may be applied in the field of pharmacotheraputics with the use of photodynamic therapy but this is not required. Sports Medicine has developed a major need for this type of product for the service of patients, of all age groups, acquiring the need for immediate and/or long-term controlled noninvasive, noncoherent radiant heat therapy specific to the inflammation of joints, tendons and ligaments. Most common needs are associated with arthritis, sprain, strains, tears, blunt trauma and orthopedic surgery of the joint membranes or the loss of damaged cartilage.

The goal of the herein disclosed invention is to improve the outcome of the treatment by shortening the period of edema and tenderness and muscular atrophy in the local area and surrounding tissue.

References Cited to Show the State of the Art

A method of treating skin injuries found on the human body (Russian Patent No. C1 2032432 Apr. 30, 1995) is known based on the effect produced by a pulsed monochromatized light beam in the red wavelength band. The beam pulse mode, however, is applied in a limited wavelength band as the treated tissues are exposed to light having the wavelength of only 0.6 to 0.69 mu.m at a reduced power density of 5 to 10 mW/cm.sup.2. Thus, it can not produce a curing effect for the whole class of diseases accompanied by metabolic disorders.

A design for multi-wavelength medical laser (U.S. Pat. No. 5,304,167 Apr. 19, 1994) is known that generates a first beam of pulsed electromagnetic energy and a second beam of electromagnetic energy having its wavelength in a visible portion of the optical spectrum, with both of them affecting the tissues simultaneously. This reference, however, discloses that the laser's wave energy is used for surgery rather than therapy.

A method of stimulating biologically active points (Russian Patent No. 93003767 A, Jul. 27, 1995) is known that stimulates body processes through use of IR-range wavelengths that feature a better penetration through the skin. However, the irradiation waveband ranges from 0.8 to 3 mu.m with its source located over the biologically active points affecting the entire body functions, rather than over the organ that controls the course of the disease, thus leaving the disease out of consideration.

High energy light emitting diodes (LED's) for photodynamic therapy (PCT Patent No. 93/21842 A1, 1993) are known. The device and the method suggested for activating the healing processes by photodynamic therapy utilize the emission of powerful LED's in a certain pre-selected portion of the optical spectrum. However, a complex feedback circuit needed to monitor the light parameters makes it impossible to adjust the device to specific type of disease.

A light therapy system (U.S. Pat. No. 5,259,380 Nov. 9, 1993) is known based on LED's that emit a narrow-band non-coherent light with a central wavelength. The LED's are grouped into diode banks controlled by a device that generates a difference of potentials and a unit that forms a voltage with preset characteristics. However, selection of required emission parameters is performed by the entire system, rather than through use of emitter properties.

The proposed device (JITMon) designed by the inventors is different in many ways. First, the JITMon is in direct contact with the skin and not spaced above the patient. Second, the JITMon device has continuous monitoring of both external tissues and internal tissues surrounding the injured site. When these areas reach a certain temperature, the device will recede into a “rest mode”. This mode of operation will allow for the temperature of the tissues to go back to normal and also allow for better hydration in the area of the injury, eliminating the secondary problem of inflammation or dehydration. Third, the material that will be used in the design of the JITMon device is designed to allow moisture to evaporate and air to penetrate naturally to the skin surface; this will act as a natural cooling mechanism needed for the promotion of healing. Fourth, by the use of a broader spectrum of light emitting diodes, the device is not limited to just IR-A emissions but will have a full range of light working from the dermal layers of the skin to the intramuscular and skeletal tissues.
(German Patent #DE4112275, 1992-11-19)

In this patent, Dr. Zetterer has invented a device allowing for the use of optical filters implementing splitting of colors, in the use of Photo-Dynamic Therapy (PDT). This technology is in use today. It allows for the use of radiation to be applied to a tumor within the human body. Today this device is used within many hospitals and is generally known as “Radiation Treatment”. It is applied with a laser and the accuracy is well determined.
(German Patent #DE4129192, 1993-03-04)

This patent describes the use of reflective radiation (irradiation) by using the composition of concave mirrors and semi-permeable mirror technology. This technique will allow the light of a wavelength to be focused to a specific location (similar to using a magnifying glass and a sun beam onto a piece of paper). Using this technique, allows for the “reduction of weight and dimensions and/or facilitates use of stronger light without overloading the cuvette”.

Because of the close proximity of the light source(s) found in our JITMon device(s) to the subject (patient), using concave mirrors for focal infusion of the wavelength(s) is not used. This is a major improvement in the overall technology because of the controllability of the wavelengths and the improvement to LED technology in today's commercial market.
(Russian Document ID(s) 93003767A, 93015098A, 94019587A)

These three documents were established in the period of 1995 through 1997 and would be considered similar to our approach. Fundamentally, all three of these documents identify using IR technology in the physiology of the human body, as does our patent. The major differences that we have noticed is that the wavelength and duration of irradiation are not controlled by automated processes and that the wavelengths and irradiation wavebands are fixed in an “all ON” or “all OFF” condition, where the JITMon Device will allow for pulsed irradiation and controlled temperature to a site working from the center (skeletal) of the inflamed area out to the dermal (skin layers). In addition, the JITMon Device will be able to be worn like an “ACE Bandage” or brace while in an active mode.

Recently issued U.S. patent documents wherein the phototherapy device is applied directly to or worn on the patient's body are as follows:

Zharov in U.S. Pat. No. 6,443,978 teaches a device for physiotherapeutic irradiation by light with the help of a matrix of various sources of optical radiation such as lasers or light diodes placed on the surface of a substrate whose shape is adapted to the shape of the zone of pathology to be treated. The device can be fixed to the patient-treatment area. Modules are provided to adjust the temperature, pressure, gas composition over the pathological area. Application of light-therapy to treat various pathologies on the bio-object's surface including dermatology, cosmetology; the treatment of traumas, bruises, oedemas, varicose veins, blood therapy and the treatment of infectious processes is disclosed. Further in the Zharov Device, the LED's are to be placed surrounding the injured site. “An oblong hollow cylinder with the light diodes placed on the side, with output windows turned inside the cylinder. The form of the internal surface depends on the bio-object's shape and can be close to cylindrical, conic or their combination.”

On the other hand in the JITMon Device, to help diffuse the light source from the LED's, a thin cellophane film with a lightly transparent colored hue is integrated into the design of the JITMon, located at the skin contact layer of the device. This single thin layer allows for light diffusion to occur and for the ability of the JITMon Device to apply controlled irradiation to the injured site. It acts in a manner similar to using photosensitizers without having to inject colloidal suspensions into the skin/muscle tissues. In addition, a thin clear ointment to the skin may also be applied with metal compounds to act as an additional diffuser if needed.

As a further distinction with the JITMon device, the LED's are placed in a thin, wafer-like non-allergenic envelope. These strips are placed in multiple layers integrated into a neoprene type material of no more than 3.5 cm from the skin. They are flexible and have limited stretching capabilities within the design of the device(s). The multiple layers are an important feature not shown by Zharov.

Rosen (U.S. Pat. No. 6,045,575) is for an apparatus for treating neonatal jaundice in the form of a garment which has semiconductor light sources affixed thereto for radiating toward the “inside” of the garment with the infant being dressed in the garment. Further, Rosen does not mention the use of light emitting diodes.

Russell (U.S. Pat. No. 6,290,713) is directed to a phototherapy device having at least one light-generating source on a flexible substrate which can be worn on various parts of the body. The illuminators can be provided with a means for cooling the heat produced by the heating elements. The Russell device is but a single layer light source and is not multiple-layered as the JITMon Device.

Vreman (U.S. Pat. No. 6,596,016) teaches a phototherapy garment containing light emitting diodes positioned within the garment. Not taught by Vreman is the concept of a multiple layered array of LED's allowing for a variety of wavelengths and intensities.

Prescott (U.S. Pat. No. 5,616,140) teaches a battery operated, portable laser bandage having one or more lasers or hyper-red light emitting diodes. The device is to be worn by a patient and in addition the device may be programmed with a laser therapy regimen. Unlike the device of this invention, the Prescott device is primarily a heat-producing device.

None of these U.S. patents teaches the concept of a phototherapy device having multi-layered Light Emitting Diodes (LED) with each layer of LED's having its unique spectral power range (e.g., wavelength) and modulated light frequencies. Nor does the prior art teach the unique method for controlling the light source and monitoring the treated site.

The device of the herein disclosed invention has the following features and objectives which allow for most efficient administration of heat and light therapy.
1) The device has an emitter included in the circuit design for varying pulse cycles of the operating mode for the device.
2) The device allows for the optimum combination of emission characteristics to obtain the maximum curing effect for a specific disease.
3) The device can combine polarization and modulation of light together with power and wavelength variation to obtain an optimum combination of emission parameters for treating a specific disease.
4) The device (JITMon) is in direct contact with the skin and not spaced above the patient.
5) The device is capable of continuous monitoring of both external and internal tissues surrounding the injured site.
6) Temperature is a factor programmed into the device to control photo-output.
7) The device is fabricated with a material which will allow skin-evaporation of moisture along with air penetration.
8) The device uses a broad spectrum of light emitting diodes working from dermal layers of the skin; with this array of light emitting diodes working from dermal layers of the skin to the intramuscular and skeletal tissues, efficient phototherapy will be accomplished.
9) The device will have wavelength and duration of irradiation controlled by automated processes; the irradiation may be pulsed; temperature controlled (internally, skeletal) and external (dermal-skin).
10) The device is designed to be worn by the patient for short or long periods of time.
11) The device provides for strategically located Light Emitting Diodes (LED's) with calibrating wavelengths and modulated light frequencies to allow for controlled heat/energy, using optical fiber, and photodetector and photoresin technology.

### SUMMARY OF THE INVENTION

The herein disclosed invention is directed to the design and construction of a device or devices to be used in methods of light and heat stimulation of human and animal tissue, allowing for the reduction of inflammation and edema to joints, tissue and nerve bundles associated with trauma. This device consists of implementation of light-emitting diode technologies such as modulated light frequencies and wavelengths but is not limited to this technology. In addition, the design incorporates medical feedback, custom software programming and engineering allowing for diagnostic interpretation, biomedical recording and patient statistical/historical medical events in “real time” mode. This will allow for data to be transmitted via telemetry or “direct connect” to other diagnostic equipment.

By surrounding the injured and inflamed areas with an elastic fitted device, which applies controlled heat/energy using Light Emitting Diodes, (LED's) at specific modulated light frequencies and wavelengths, the recovery process will be enhanced. Understanding the anatomy and physiology of the body and its process to healing and helps the physician or therapist to apply proper heat/energy where needed. This in turn improves blood flow and enhances the natural release of cells and chemicals to improve the overall recovery of the patient. Each device will be made to specifically fit the areas of the joints and skeletal system especially the neck, thoracic, knee, elbow and tarsal and carpal appendages. The device will have the ability to integrate optical fiber, medical sensors and photoresin technology, solid state detectors, sonic/ultrasonic transducers or other high level inputs.

More specifically, the herein disclosed invention is involved with a device and a method for treating and reducing inflammation and edema both internal and external, to joints, muscles, nerves and skin tissues of the subject (human or animal) comprising, an elastic, portable device configured to be worn in contact with the skin and surrounding the area or areas of inflammation, edema, neural and muscular damage over short and long periods of time; whereas the construction of the device is configured with multiple layers of LED's and fiber optics distributed in a range consisting of “near contact with the skin to a few centimeters from the skin tissue; with orientation toward the subject; integrated low voltage power. Electronic memory and communications via analog/digital connection or telemetric medical sensor; allowing for independent control of tissue temperature and modulation of the light frequencies and wavelengths of the LED's is provided.

The preferred device of this invention is a device having multi-layer (or multi-tiered) light emitting diodes (LED's). The preferred number of layers is either three or four with the preferred number of layers being four. While not preferred, the device can be produced with a single layer of LED's and fiber optics. The inventors do not want to be limited to only four layers, since more layers are possible as understood by those skilled in the art. The device is to be configured to have multiple types and modulated light intensities of LED's in multiple ranges along with fiber optics. As an alternative the device may be configured with multiple types and modulated light intensities including Laser Diode Technology and fiber optics. A single layer of modulated light intensities including Laser Diode Technology, fiber optics and diagnostic feedback is also contemplated.

Viewed in another aspect, the present invention provides in an apparatus for diagnosing and/or treating a patient's medical problem with light therapy, the improvement which comprises a multi-tiered light source, at least one sensor for monitoring a condition of the patent as the light therapy is applied to the patient, and a feedback loop responsive to the sensor for adjusting the multi-tiered light source for optimizing the light therapy applied to the patient. Preferably, the multi-tiered light source comprises a plurality of LED's; and the frequency of the plurality of LED's is adjusted by the feedback loop, the multi-tiered light source having different frequencies.

In a preferred embodiment, the sensor monitors temperature of the patient's skin.

Alternatively, the sensor monitors oxygen levels of the patient's skin tissue.

The feedback loop may adjust the pulse width and/or the rep rate of the light therapy.

Distance

(3-Tier Design)

The LED's are to be enclosed in a wafer-like strip(s). Each strip would be symmetrically positioned and associated with a geometric plane. For instance, in a three-tiered device, the 250 nm to 500 nm strips would be positioned within the neoprene envelope on the internal (medial) surface of the neoprene device and within contact or near contact with the human tissue. The 500 nm to 700 nm wafer-like strip(s) would create the next level or middle level of LED's being separated by about 0.1 mm in depth from the first level. Finally, the third level would consist of LED's ranging from 700 nm to 20,000 nm and being separated from the middle level by at least about 0.1 mm and no greater than about 20 mm in depth from the level associated with skin contact.

(4-Tier Design)

The LED's are to be enclosed in a wafer-like strip(s). Each strip would be symmetrically positioned and associated with a geometric plane. For instance the 250 nm to 500 nm strips would be positioned within the neoprene envelope on the internal (medial) surface of the neoprene device and within contact or near contact with the human tissue. The 500 nm to 700 nm wafer-like strip(s) would create the next level or middle level of LED's only being separated by about 0.1 mm in depth from the first level. The third level would consist of LED's ranging from 700 nm to 900 nm also being separated by at least about 0.1 mm and no greater than 5 mm in depth from the second level. Finally, the fourth level would consist of LED's ranging from 900-20,000 nm also being separated by at least about 0.1 mm and no greater than 20 mm in depth from the third level.

The device lends itself to the development of software to be integrated into a personal computer or a hand held device allowing for the monitoring and documentation of information accumulated from identifying wavelengths, light modulated frequencies, localized heat and heat variances, skin temperature and other biometrics as needed associated with the subject as it applies to the location of the device.

Further, the integration of laser photo diodes and photodetector technology allows for data to be gathered stored and retrieved in both “real time” and historical events. This integration is to be coupled with PMT technology and CCD Technology.

The device is to be constructed of single or multiple layer technology integrating the light emitting diodes (LED's), fiber optic strands and fiber optic bundles, light guides and polarization optics.

The device is to be constructed of a neoprene type material or other Non-Allergenic Material(s), allowing for elasticity, flexibility, protection and comfort of the injured site of the patient, and will be designed in multiple pediatric and adult sizes i.e. small, medium, large and extra-large and with wrap-around Velcro® type adhesive/connectivity or other connective material allowing for a secure fit.

The device is designed with LED's or Laser Diodes having wavelengths in ranges of 250 nm to 20,000 nm. The LED's or Laser Diode wavelength range of 250 nm to 20,000 nm will be introduced to the skin tissue allowing for muscular and or neural stimulation under low light conditions. The modulated light frequencies in a range of less than (<) 1 Hz and less than (<) 1 GHz will be introduced to the skin tissue allowing for muscular and or neural stimulation under low light conditions. The LED's or Laser Diode wavelength range of 250 nm to 20,000 nm will be introduced to the skin tissue allowing for internal penetration of the skin tissue inducing controlled heat/energy throughout the injured area

The device has controlled penetrating light wavelengths and modulated light frequencies using light-emitting diodes to control heat/energy and duration directly to the injured site.

The Joint/Tissue Inflammation Therapy and Monitoring device of the invention is a phototherapy device for light and heat treatment and for reducing inflammation, edema and/or medical conditions associated with the joints, muscles, wound healing, nerves and skin tissue of a human or animal subject comprising, a portable device configured to be worn in contact with the skin and surrounding the area or areas of pain, inflammation, edema, neural and/or muscular damage. The device is configured to have multiple layers of LED's having varying wavelengths with the multiple layers of LED's being distributed in a range of near contact with the skin tissue to a few centimeters above the skin tissue. The device will have LED's having a light spectrum from ultraviolet to near infrared. The device will be configured with multiple layers of LED's having wavelengths in the range of 250 nm to 20,000 nm. In one embodiment of the device it will be configured with three layers wherein the LED layer closest to the skin has a wavelength of 250 nm-500 nm; the next level has a wavelength of 500-700 nm and that furthest from the skin has a wavelength of 700-900 nm. In another embodiment there will be a four-layer device wherein the LED layer closest to the skin has a wavelength of 250 nm-500 nm, the second level has a wavelength of 500-700 nm, the third level has a wavelength of 700-900 nm and the fourth level has a wavelength of 900-20,000 nm. More specifically, the LED's working mode is a CW light mode, modulated light mode, or pulsed light mode. The device can be configured with multiple LED's having light wavelengths of 250 nm to 20,000 nm and modulated or pulsed light frequencies of 1 Hz through 1 GHz. The phototherapy device can be further provided with integrated low voltage power, electronic memory and communications via analog/digital connection or telemetric medical sensor; allowing for independent control of tissue temperature and operating and resting (“rest mode”) device working mode, modulation of the light frequencies, intensities and wavelengths of the LED's. In addition, the device is provided with multiple types of CW or modulated light intensities of LED's of multiple spectral ranges and fiber optics.

The device is designed to provide multiple types and CW or modulated light intensities including Laser Diode Technology of multiple spectral ranges and fiber optics to read physiological and biochemical changes within the area or areas of inflammation, edema, neural and/or muscular. Software is provided to be integrated into a personal computer or hand held device allowing for the monitoring and documentation of information accumulated, identifying wavelengths, light intensities, light modulated frequencies, localized heat and heat variances, physiological and biochemical changes, skin temperature and other biometrics as needed associated with the subject as it applies to the localization of the device. The device is further provided with the integration of laser diodes and photodetector technology allowing for data to be gathered stored and retrieved in both “real time” and historical events coupled with PMT Technology, Spectral Technology and CCD Technology. The device is designed to have multiple layers of LED's and fiber optics have multiple types of modulated light intensities of LED's of multiple ranges and fiber optics distributed in a range of near contact with the skin to a few centimeters from the skin tissue; with orientation toward the subject.

The device integrates multiple layer technology integrating the light emitting diodes (LED's), laser diodes, fiber optic strands fiber optic bundles and polarization light optics. The body of the device is constructed of a neoprene type material or other non-allergenic material(s), allowing for elasticity, permeability, flexibility, protection and comfort to the injured site of the patient, and is designed to accommodate multiple pediatric and adult sizes; i.e., small, medium, large and extra-large and with wrap-around Velcro® type adhesive/connectivity or other connective material allowing for a secure fit, for cosmetic purposes with multiple construction designs of elastic portable device for different body parts. The device is provided with Light Guide Technology for uniform illumination of the skin and surrounding the area or areas of inflammation, edema, neural and/or muscular damage over short and long periods of time.

The invention envisions a method comprising applying to the skin of a patient phototherapy with a device configured with multiple layers of LED's having varying wavelengths and fiber optics such that the patient or a healthcare professional will be able to choose a therapeutic treatment from one or all of the following ranges,
a) a first range of 250 nm to 500 nm to penetrate the dermal and sub-dermal tissue allowing for a stimulation of blood cells, vitamins, proteins, molecular genetic material, amino acids, enzymes and other physiological mechanics to be implemented for the reduction of edema and discoloration to the injured site,
b) a second range of light, of 500 nm to 700 nm for deeper non-invasive penetration of the tissue into the structure of the muscles, fibers and tendons,
c) a third range or layer of light, of 700 nm to 900 nm allowing for the penetration of tissue, down to the skeletal structure for stimulating the natural process of healing by invoking physiological processes to improve the reduction of edema, stimulation of blood circulation, reduction of neural edema, regeneration of tissue and improved enzymatic processes, and
d) diagnostic therapy will be transitive to polarized or non-polarized light.

In another embodiment the invention envisions a method comprising applying to the skin of a patient phototherapy a device configured with multiple layers of LED's having varying wavelengths and fiber optics such that the patient or a healthcare professional will be able to choose a therapeutic treatment from one or all of the following ranges,
a) a first range of 250 nm to 500 nm to penetrate the dermal and sub-dermal tissue allowing for a stimulation of blood cells, vitamins, proteins, molecular genetic material, amino acids, enzymes and other physiological mechanics to be implemented for the reduction of edema and discoloration to the injured site,
b) a second range of light, of 500 nm to 700 nm for deeper non-invasive penetration of the tissue into the structure of the muscles, fibers and tendons,
c) a third range or layer of light, of 700 nm to 900 nm allowing for the penetration of tissue, down to the skeletal structure for stimulating the natural process of healing by invoking physiological processes to improve the reduction of edema, stimulation of blood circulation, reduction of neural edema, regeneration of tissue and improved enzymatic processes,
d) a fourth layer of light furthest from the skin in the range of 900 to 20,000 nm, and
e) diagnostic therapy will be transitive to polarized or non-polarized light.

In the invention device non-light related heat generated from the device electronics including LED's and laser diodes is used as a part of phototherapy.

The LED's in each tiered group of the JITMon device could be programmed to be turned on and off as a unit or each tier could be turned “on” or “off” based on the physiological or therapeutic requirements of the patient. Thus, there could be discretion as to the specific light wavelengths to be used to treat a specific patient-condition.

The following abbreviations and terminology being used herein are defined as follows:
WAVELENGTH Light being measured in nanomenters (nm).
FREQUENCY Light being measured in Hertz, Mega-Hertz and Giga-Hertz.
PMT Photomultiplier tube (PMT) is an electronic detector that measures the intensity of faint light. It is a vacuum tube that is as old as color television. PMTs are very competitive with semiconductor-based sensors and outperform them in many applications.
VDC Voltage Direct Current
CCD Charge-Coupled Device
EEPROM Electronic Erasable Programming Read Only Memory
VAC Voltage Alternating Current
CPU Central Processing Unit
EEPROM Eraseable Electronic Programming Module
A/D I.O. Analog/Digital Input Output
USB Universal Serial Bus
PDA Device Personal Data Access Device, i.e., Palm Pilots Axim, Clie, Pocket PC and other hand-held storage devices
VDC Technology Voltage Direct Current Technology

In general, the dimensions of the Light Emitting Diodes (LED's) can be quite small measuring about 1 mm×1 mm×1 1/2-2 mm. LED's are conventional in the phototherapy art and are described in the prior art references cited herein.

The device is to be produced with the following features:
1) The device is designed with insulated low voltage being produced by VDC Technology;
2) The device will incorporate a micro circuit board containing an Electronic Erasable Programming Read Only Memory (EEPROM) chip, Central Processing Unit (CPU), CCD Integration, Laser Photo Diodes, Photodetector Technology, PMT biometrics sensory devices and digital input/output device;
3) The device will incorporate a telemetric monitoring transceiver configured for Spread Spectrum Technology in bandwidths ranging in 2.0+Ghz or others approved by the FCC;
4) The light-emitting diodes or laser diodes will be integrated into the device using all wavelengths of the spectrum and modulated light frequencies for less than (<1 Hz to less than (<) 1 Ghz, as possible, for the technology;
5) Sensory devices will be integrated into the device allowing for physiological monitoring of the patient and for the ability of adjusting the wavelength and modulated light frequencies;
6) The device will be configured with LED's in multiple layers consisting from depths of contact with the skin and continuing to 3.5 cm or more;
7) The device will be configured with arrayed holding devices in single, tri-level and quad-level configurations allowing for multi-band LED's to be connected to fiber optic cabling encapsulating the skin tissues at multiple points separated as needed or required to produce optimal results;
8) The device will be designed and configured for the light guide technology wavelength in the spectra of Ultra Violet, Blue, Green, Yellow, Red and Near Infrared;
9) The device will be configured with LED's in single layer(s) consisting of depths from contact with the skin and continuing to 2 cm or more;
10) The device will be configured with an array, holding devices in single, tri-level and quad-level configurations allowing for multi-band LED's to be connected to fiber optic cabling encapsulating the skin tissues at multiple points separated as needed or required, to produce optimal results;
11) The device will be configured with multiple types and ranges of LED's consisting of depths from contact with the skin and continuing to 2 cm or more;
12) The device will be configured with an array, holding devices in single, tri-level and qua-level configurations allowing for multi-band LED's to be connected to fiber optic cabling encapsulating the skin tissues at multiple points separated as needed or required, to produce optimal results;
13) The device will be configured with multiple layer configurations implementing Laser Diode Technology consisting of depths from contact with the skin and continuing to 2 cm or more;
14) The device will be configured with Laser Diode Technology to be connected to fiber optic cabling contacting the skin tissues at multiple points separated as needed or required, to produce optimal results;
15) The device will be configured with an array, holding devices in single, tri-level or four-level configurations allowing for laser diode(s) to be connected to fiber optic cabling encapsulating the skin tissues at multiple points separated as needed or required, to produce optimal results. The number of levels can be varied to more or fewer than four and wavelengths adjusted accordingly as would be understood by those skilled in the art;
16) The device will be configured with single layer technology implementing Laser Diode Technology consisting of depths from contact with the skin and continuing to 2 cm or more;
17) The device will be configured with Laser Diode Technology to be connected to fiber optic cabling encapsulating the skin tissues at multiple points separated as needed or required, to produce optimal results;
18) Biometrics sensors will monitor induced heat/energy, blood flow, baseline temperatures, oxygenated blood and circulation;
19) The device is designed to be used with a photosensitizing agent or without, depending on the need and recommendations of the physician;
20) The device will be designed to have a reflective property or with a clear film allowing for refractive wavelengths of LED's surrounding the injured site;
21) By applying these devices to an injured joint, a non-invasive method will be established to decrease inflammation and edema and to enhance the recovery time and flexibility of the joint and damaged tissues. This will allow for a better prognosis and implementation plan by the physician;
22) Software and engineering designs will interpret and record the diagnostic information;
23) Proprietary software is designed and compiled to integrate to the micro circuit board, CCD Technology and EEPROM previously identified;
24) Telemetry and network engineering will allow for communications between the device and information repository (personal computer, CCD or Hand Held Device);
25) The device will have an integrated Digital Input/Output Device allowing for direct connectivity between the information repository and device allowing for calibrations and documentation of changes introduced to the device.
26) In the phototherapy device, LED's have a working mode which is a CW light mode, modulated light mode, pulsed light mode. In a specific embodiment, the device is configured with multiple LED's having light wavelengths of 250 nm to 20,000 nm and modulated or pulsed light frequencies of 1 Hz through 1 GHz. The devise may have integrated low voltage power, electronic memory and communications via analog/digital connection or telemetric medical sensor; allowing for independent control of tissue temperature and working mode, modulation of the light frequencies, intensities and wavelengths of the LED's as in alternative phototherapy device may be provided with multiple types of CW or modulated light intensities of LED's of multiple spectral ranges and fiber optics, as well as, Laser Diode Technology of multiple spectral ranges and fiber optics. Means are provided to produce multiple types and CW or modulated light intensities including Laser Diode Technology and fiber optics to read physiological and biochemical changes within the area or areas of inflammation, edema, neural and/or muscular.

As used herein: Wavelengths are measured in nanometers (nm); frequencies are measured in Hertz (megahertz, gigahertz, terahertz . . . ). Both of these values combined are usually identified as the “Visible Light Spectrum” or electromagnetic waves.

### BRIEF DESCRIPTION OF THE DRAWINGS

FIG. 1 is a plan view of a prior art therapeutic laser treatment device.

FIG. 2 is a side view of the prior art device with part of the cover broken away to schematically show that the vertical-cavity surface emitting laser (VCSEL) are on a single plain to be applied close to the skin. (Note this is a different technology than what the herein disclosed invention is providing. The design in FIG. 1-2 is based on quasi monochromatic light with heat sinking. This technology is LASER not LED.)

FIG. 3 is a perspective view of the Joint/Tissue Inflammation Therapy and Monitoring Device for application to the knee.

FIG. 4 is a top plan view thereof.

FIG. 5 is a cross-section schematic representation of the JITMon Device four-tier design taken along lines 5-5 of FIG. 4.

FIG. 6 is a cross-section representation of the JITMon Device three-tier design shown with fiber optics.

FIG. 7 shows an alternative design cross-section JITMon Device four-tier design Concept Drawing with fiber optics, showing a limited function for the fiber optics.

FIG. 8 is a figure depicting a physician, in a remote location from the patient, interacting with the Joint/Tissue Inflammation Therapy and Monitoring Device by remote electronic control.

FIG. 9 shows the device as would be applied to the finger.

FIG. 10 is a cross section thereof taken along lines 10-10 of FIG. 9.

FIG. 11 is a cross-section representation of the JITMon Device three-tier design with a light filter attached.

FIG. 12 is a perspective view of an alternative embodiment of the Joint/Tissue Inflammation Therapy and Monitoring Device for application to the knee and involves the use of polarized film or a light guide.

FIG. 13 is a cross-section representation thereof taken along lines 13-13 of FIG. 12.

FIG. 14 is a cross-section representation thereof taken along lines 14-14 of FIG. 12.

FIG. 15 is a view showing a knee JITMon Device and the internal arrangement of LED's along with LED fiber optic strips and processor.

FIG. 16 is an enlarged portion thereof.

FIG. 17 is a view showing the JITMon Device as would be applied to the wrist and hand.

FIG. 18A and 18B are a detail cross section of a JITMon Device with laser diode and fiber optic circuitry.

FIG. 19 is a simple schematic representation of the components of the device as would be used for optimum light therapy.

FIGS. 20A-20B are a computer program flow diagram for programming the phototherapy device of the invention.

FIG. 21 sets forth ancillary equipment to be used with the JITMon Device.

### DETAILED DESCRIPTION OF THE INVENTION

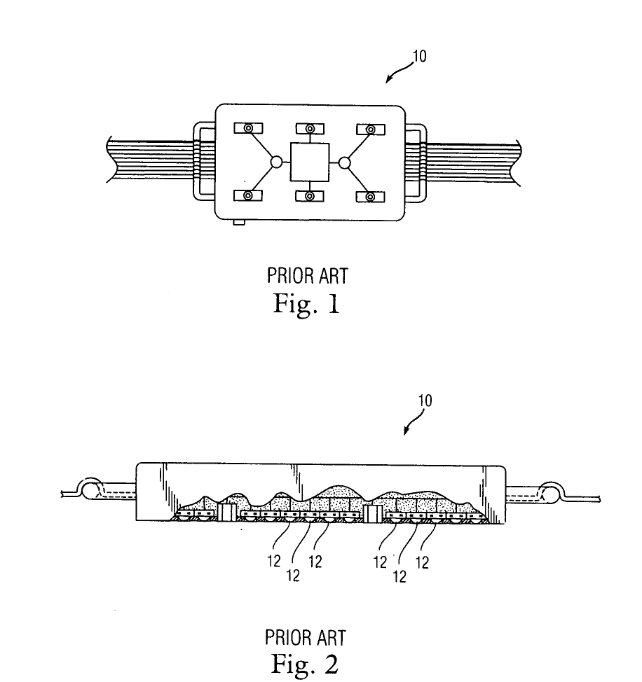

With reference to FIG. 1, the prior art device 10 is a battery operated laser treatment device having therein lasers and hyper-red light emitting diodes embedded in a bandage to be worn by a patient. As opposed to the tiered therapy device of the herein disclosed invention, the vertical-cavity surface emitting laser (VSCEL) 12 are on a single plain and are quasi-monochromatic.

Referring to FIG. 2, there is shown a side plan view of the prior art device 10 with part of the outer cover broken away to show that vertical-cavity surface emitting laser (VCSEL) 12 are on the same level and therefore would be the same distance from the skin.

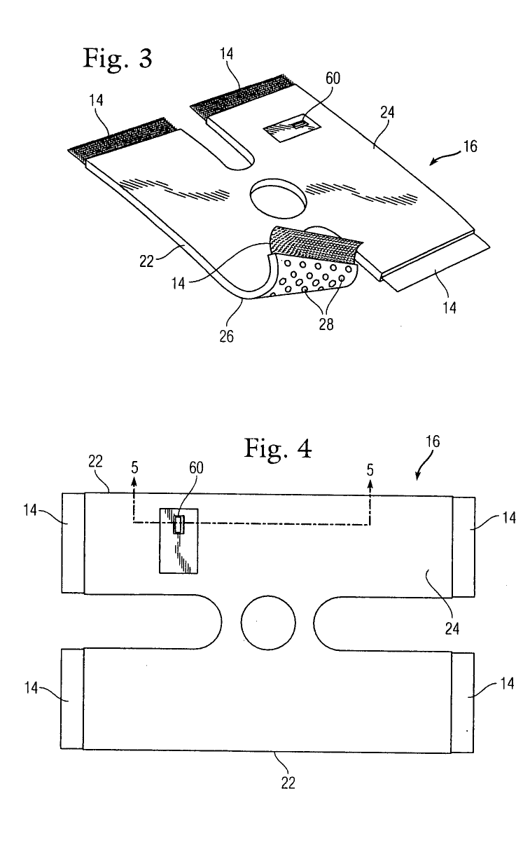

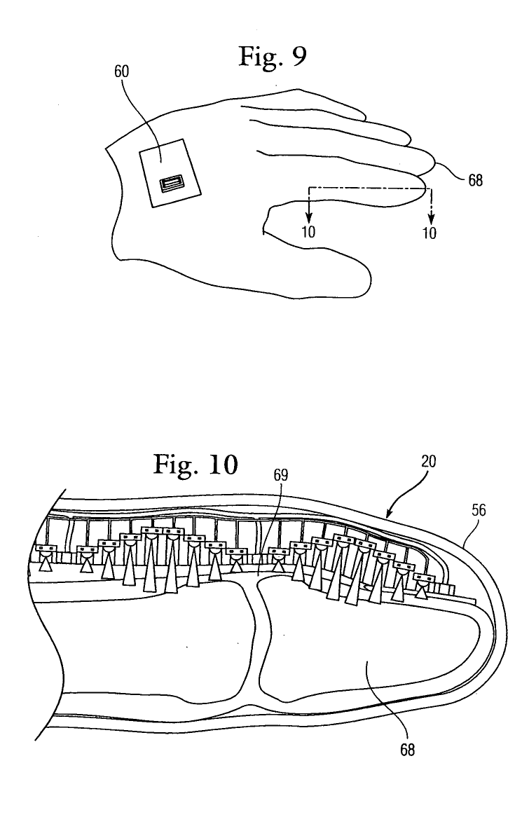

FIGS. 3 and 4 are views of the Joint/Tissue Inflammation Therapy and Monitoring Device 16 intended for application to the knee. The device 16 is generally made of neoprene 22 and has a top layer 24 and a bottom layer 26 which has disposed therein holes 28 through which the light of the light emitting diodes will pass. On the ends of the JITMon Device 16 are Velcro™ tabs 14 to be used to fasten the knee-device 16 to the knee. It is to be noted that FIGS. 3 and 4 depict the Joint/Tissue Inflammation Therapy and Monitoring Device 16 as representative of like devices to be applied to other parts of the body as for example see FIGS. 9, 10 and 13.

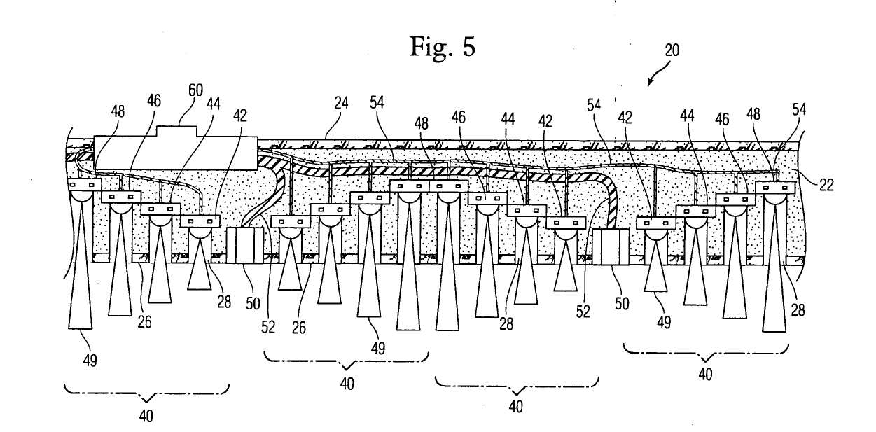

FIGS. 5-7, show the Joint/Tissue Inflammation Therapy and Monitoring Device cross-section or general concept displaying light emitting diodes (LED's) and fiber optic bundles, constructed with a neoprene (non-allergenic material) envelope creating the final device. Photo detectors and photo resin may be incorporated into the device. The figures depict typical arrangements of the tiers of the light emitting diodes.

Note particularly for example, FIGS. 5-7, and 11 depict light 49 projected from the light emitting diodes.

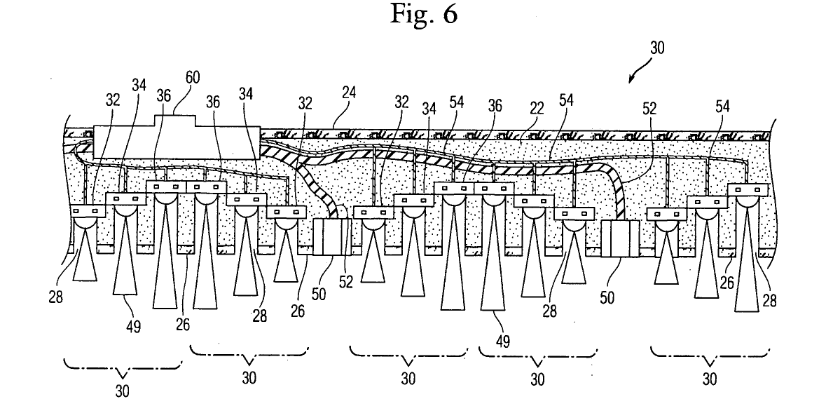

More specifically, FIGS. 5-7 are schematic representations of the multiple layered Joint/Tissue Inflammation Therapy and Monitoring Device of this invention showing modulated light in four layers or tiers shown as LED strips with light frequencies in the range of 250 nm to 20,000 nm. The device can be constructed with fiber optic bundles, biomedical feedback sensors, data processor with a data processing circuit; and laser diodes with photodetector technology along with biofeedback sensors integrated with PMT technology. Neoprene or other non-allergenic material is provided to surround the electronic components, with the final layered product to be applied to the skin. The neoprene layer next to the skin is provided with openings 28 through which the modulated light exits 49 (note cones of light) to affect the specific part of the body or condition being treated. Note that for example in FIG. 5, the modulated light sources 42, 44, 46 and 48 project light shown by cones 49 coming through an opening 28 in neoprene skin-contact layer 26. FIG. 6 is similar to FIG. 5, but describes the three-layer concept of the invention.

Note particularly in FIGS. 5-7, the LED's and fiber optic sensory device 50 are provided with fiber bundles/wires 52 and 54 connected to the processor 60. Operatively, the fiber bundle 52 would carry a signal (a sensed condition) from the fiber optic sensory device 50 to the processor 60 for processing and then to indicate based on the condition sensed the specific LED's to be actuated to treat the sensed condition. Note that fiber bundle 52 would run from the fiber optic sensory device 50 to the processor 60 and wire 54 in turn would run from the processor 60 to actuate a specific LED.

More specifically, referring to FIG. 5 there is shown a cross-section taken along lines 5-5 of FIG. 4. Shown are banks 40 of four-tiered light emitting diodes along with a strategically placed fiber optic sensory device 50. The light emitting diodes are numbered 42, 44, 46, 48 respectively with light emitting diode 42 being placed in the neoprene envelope 22 so as to be closest to the skin when the inventive device is worn and light emitting diode 48 being furthest from the skin. The Joint/Tissue Inflammation Therapy and Monitoring Device 20 has a neoprene belt 22 having a top layer 24 and a skin-contacting layer 26. Embedded in the neoprene belt 22 are a series of light emitting diodes 42, 44, 46, 48 in a tiered relationship, and adjacent thereto are a tiered series of light emitting diodes and between these sets of light emitting diodes is a fiber optic sensory device 50. Disposed on the top layer of the belt is a processing unit 60. In FIG. 5 the device could be programmed so that individual LED's are actuated. For example, LED 42 and 48 could be programmed to be “on” while LED 44 and 46 could be programmed to be “off”.

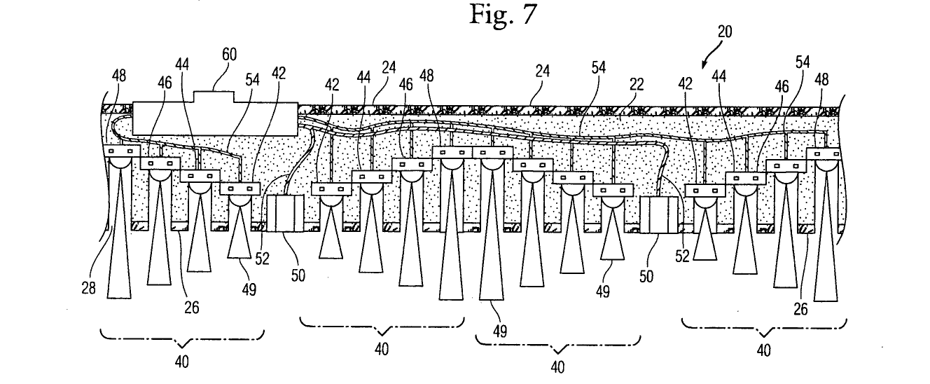

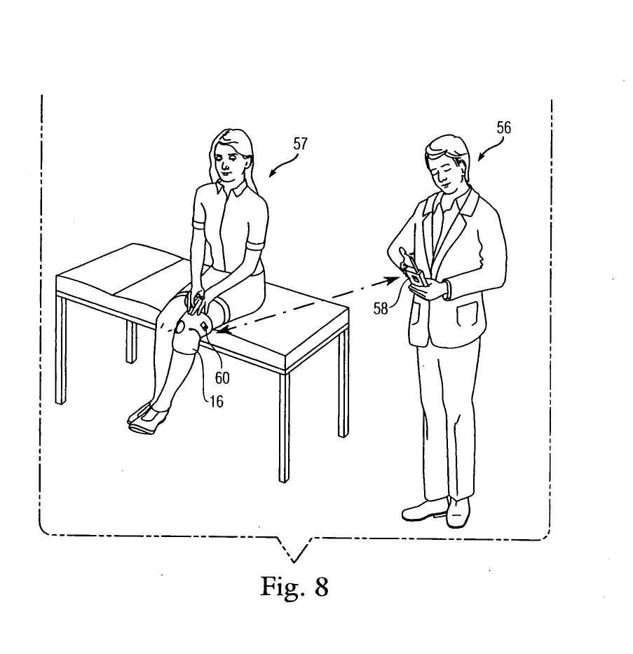

More specifically, referring to FIGS. 5 and 7 in a four-tiered Joint/Tissue Inflammation Therapy and Monitoring Device 20 of this invention is provided with modulated light in the wavelength range of 250 nm-20,000 nm. Note that modulated light coming from LED 42 which is in place closest to the skin would be blue light of wavelength 250-500 nm; the second level modulated light 44 would have a wavelength of 500-700 nm; the third level modulated light 46 would have a wavelength of 700-900 nm; and the fourth level modulated light 48 (furthest from the skin) would have a wavelength in the range of 900-20,000 nm. The device would have LED strips ranging in wavelength of 250 nm to 20,000 nm. The device would be provided with laser diodes with photodetector technology and biofeedback sensors integrated with PMT technology. The therapy device 20 is further provided with data processing circuit 60 with a 3.5-5.0 VDC power supply, CPU, LED/fiber optic connectivity integrated with PMT technology, EEPROM, telemetry, CCD integration, A/D I.O. using USB connections. Neoprene or other non-allergenic material forms the outer layer 26 applied next to the skin and is provided with openings or holes through which the modulated light approaches the body to produce its physiologic effect. Note also that there is an upper or outer layer (24) that could be made of neoprene or other non-allergenic material. This upper layer is not provided with openings to transmit light as is the layer next to the skin.

Referring to FIGS. 4 and 6, the Joint/Tissue Inflammation Therapy and Monitoring Device 20 has a neoprene belt 22 having a top layer 24 and a skin-contacting layer 26. Embedded in the neoprene belt 22 are a series of light emitting diodes 32, 34, 36 in a three-tiered relationship 30 and between these sets of light emitting diodes is a fiber optic sensory device 50. Disposed on the top layer of the belt is a processing unit 60. In the sequence of light emitting diodes note that LED 32 would be closest to the skin and LED 36 furthest from the skin.

In a three-layered design a range beginning with 250 nm to 500 nm would begin to penetrate the dermal and sub-dermal tissue allowing for a stimulation of blood cells, enzymes and other physiological mechanics to be implemented. These physiological processes will begin the reduction of edema and discoloration to the injured site.

A second range of light, consisting of 500 nm to 700 nm begins a deeper non-invasive penetration of the tissue into the structure of the muscles, fibers and tendons.

Finally a third range or layer of light, consisting of 700 nm to 20,000 nm allowing for the penetration of tissue, down to the skeletal structure and even through, again stimulating the natural process of healing by invoking physiological processes to improve the reduction of edema, stimulation of blood circulation, reduction of neural edema, regeneration of tissue and improved enzymatic processes.

All of these wavelength and modulated light frequencies can be controlled by an electronic setting establishing a circuitry of continuous irradiation or pulsed irradiation or both simultaneously. Within this circuitry, one could control the light to certain depths of tissues and control the amount of energy being invoked to a specific site.

The inventors have developed an elegant method for delivering light and heat therapy. The method involves a multiple layered or tiered approach to delivering light and heat to the body. The method involves a device in which Light Emitting Diodes are employed in a tiered fashion within the device to supply phototherapy to the body (or body area) as exemplified by the FIGS. 5-7 described herein. The device is exemplified in the figures by a three or four tiered device; however, more or fewer tiers are possible. For example, in a three-tiered device (FIG. 6) the LED closest to the skin would have a wavelength range of 250-500 nm; the next level removed from the skin would have a wavelength range of 500-700 nm; and the LED furthest from the skin would have a wavelength range of 700-20,000 nm. The casing or belt (22) in which the LED's are contained is to be made of Neoprene or other non-allergenic material. The configuration of the LED's in the multiple tiered device(s) can vary depending on a specific medical condition, i.e., there could be a reverse configuration such as 900-20,000 nm could be the first layer, i.e., closest to the skin, and 250-500 nm could be the last layer, i.e., furthest from the skin, or layers could be doubled such as 900-20,000 in the first two layers and 250-500 nm in the middle or last layers. Cones of light are identified as 49.

More specifically, with reference to FIGS. 5-7 there is shown a “Cross-Section JITMon Device layer Design”. Note that there are Light Emitting Diode (LED) strips 20 that would be located within the envelope of the device and positioned on a geometrical plane. The LED's focal point would be positioned toward the skin tissue of the subject. Note particularly the alternative placement of the light emitting diodes.

The LED's are fashioned in such a way that they would consist of light wavelengths of 250 nm to 20,000 nm. Modulated light frequencies have been previously defined as being in the range of 1 Hz through 1 GHz. Most of the activity will be in the ranges of 1 MHz to 800 MHz. That is, a light spectrum which consists of wavelengths demonstrated from the visible to near-infrared spectrum.

Attention is brought to the fact that the individual LED's within the band of tiered LED's can be individually programmed to turn on and off as a tiered group or could be programmed to turn on and off individually. For example in FIGS. 4 and 6, light emitting diode 32 could be programmed to be “on” while light emitting diodes 34 and 36 would be “off”.

The material of the overall cover for the device will be made of a non-allergenic material allowing for air circulation and allowing moisture to dissipate away from the injured site when the LED's are active. Currently, this type of material is being manufactures in sports clothing.

FIG. 8 shows that the doctor 56 who is at a location remote from the patient 57 can signal the processor 60 on the knee device 16 by radio transmitter 58 and thereby alter the parameters of treatment.

FIGS. 9 and 10 show how the device 20 may be applied to the finger 68 at the joint 69. FIG. 10 is a cross-section taken off of 10-10 of FIG. 9.

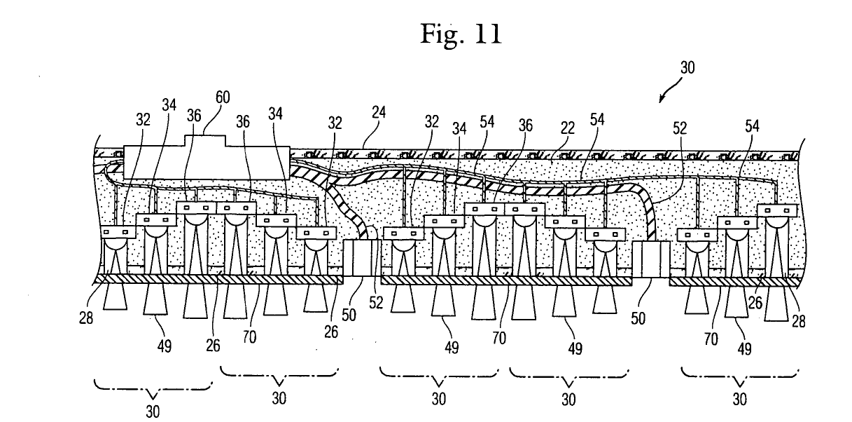

With reference to FIG. 11, a three-tier device 30 has been modified to encompass a filter 70 in this way the characteristic of light delivered can be changed simply by changing the filter through which the light shines.

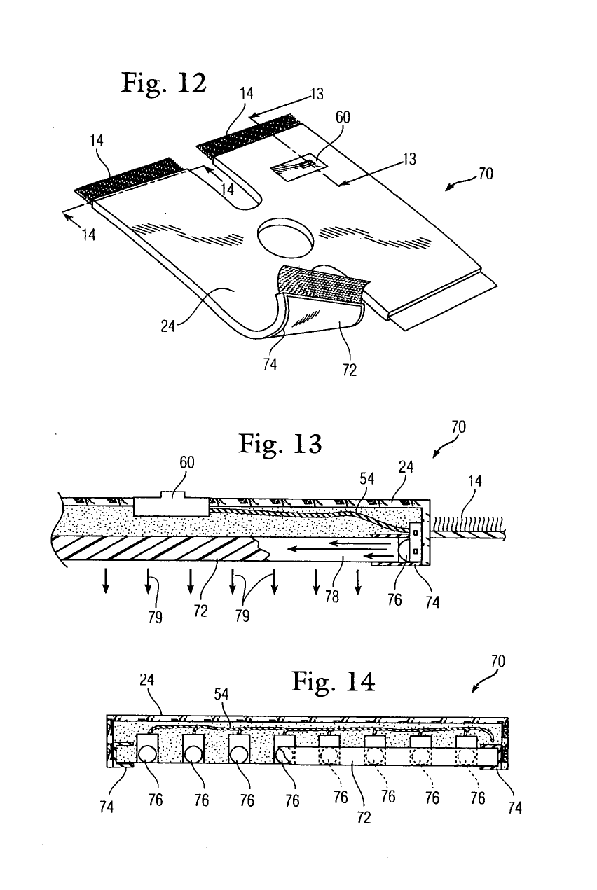

With reference to FIGS. 12-14, there is shown an alternative embodiment of a phototherapy device 70 for application to the knee. However, the device 70 can be constructed to be applied to other parts of the body in need of phototherapy. The phototherapy device 70 has therein a polarized film or light guide 72 held by a clip 74, and white light diodes 76. The white light diodes 76 shine their light through polarized film or light guide 72. The polarized film or light guide would be interchangeable and flexible, for example, be one to shine ultra-violet, blue, green, yellow, red or near infrared light. The polarized film or light guide 72 would be held in place by a flange-clip 74 to hold the polarized film or light guide in place. The device 70 would be held in place by attaching means 14, which in the figures shown is Velcro, but could be any other attaching means, e.g. adhesive, as would be understood by those skilled in the art. The outer covering of the device is neoprene or a like material. The device would have a processor 60 which would receive input information to control the light source 76. FIG. 13 is a cross-section taken along lines 13-13 of FIG. 12 and shows white light 76 entering polarized film or light guide 72 as shown by arrows 78 and exiting the polarized film or light guide as shown by arrows 79 to have the light contact the body part being treated. Light source 76 could be controlled by employing LED wire 54 leading to processor 60. It is to be understood that power (e.g., battery) could be incorporated with processor 60. FIG. 14 is a cross-section taken along lines 14-14 of FIG. 12 and shows the battery of white light diodes 76 to shine into the end of the polarized film or light guide 72. In FIG. 14 the white light diodes 76 are shown in whole lines and dashed lines to show that the white light diodes 76 are at the outer edge of the filter. While the alternative embodiment has been described in terms of white light diodes, other light sources would be applicable such as colored LED's or laser diodes passing light wavelengths through the affected area. Actually, a combination of colored lights could be used as the diodes.

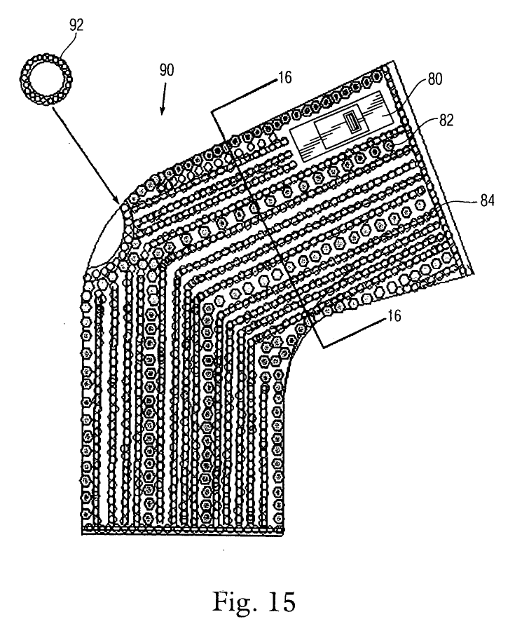

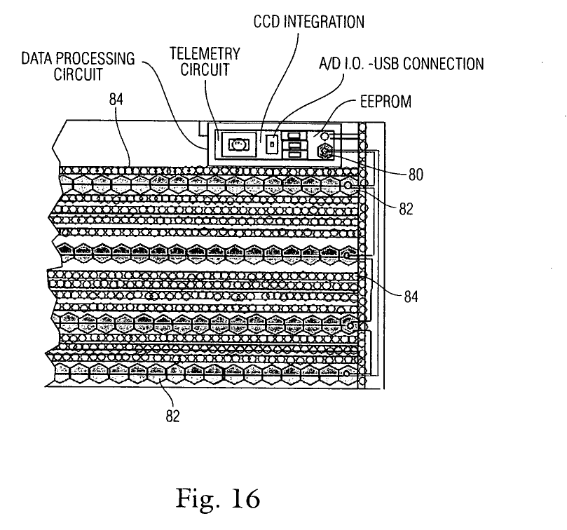

FIG. 15 shows the device 20 as applied to the knee, and FIG. 16 is an enlarged section thereof.

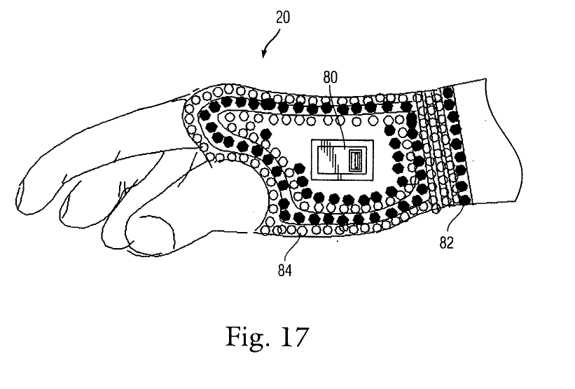

FIG. 17 illustrates the JITMon Device 20 as would be applied to the hand and wrist.

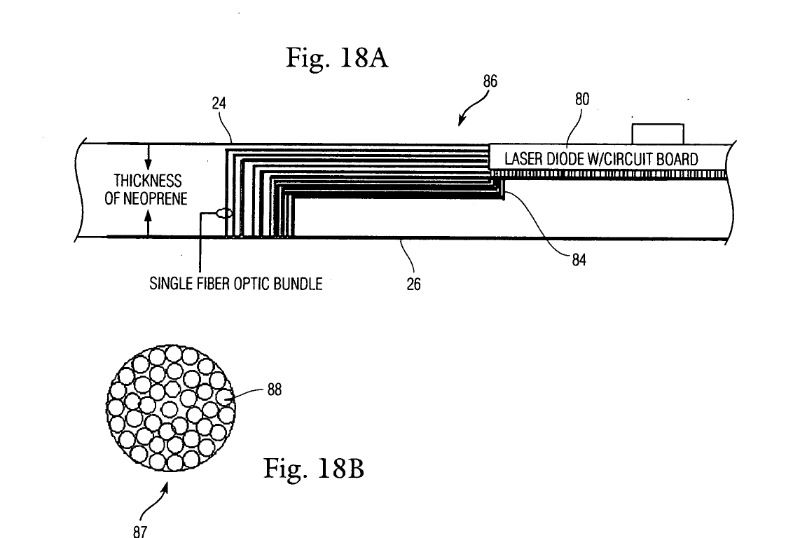

FIGS. 18A and 18B describe a schematic arrangement of fiber optic circuitry 86 as it would be within the neoprene casing; and FIG. 18B depicts a fiber optic bundle 87 containing many individual single strand 88 fibers.

More specifically, with reference to FIGS. 18A and 18B the phototherapy device 20 of this invention is shown. FIG. 18A shows fiber optic circuits and a fiber optic bundle is shown in FIG. 18B. Also shown is a single strand fiber allowing for transmission of light and data to and from the site. In the cross-section FIG. 18A there is also shown the exterior surface (away from the skin) to be made of neoprene or other non-allergenic material; data processing circuit with a 3.5-5.0 VDC power supply, CPU, LED/fiber optic connectivity integrated with PMT technology, EEPROM, telemetry, CCD Integration, A/D I.O. using USB connections; LED/fiber optic strips with wavelength control technology range 350 nm-20,000; and neoprene or other non-allergenic material edge (next to the skin) 26.

While not preferred a single layer design of this invention is possible. The single layer LED/Fiber Optic Strip of a wavelength range of 250 nm-20,000 nm; single layer laser diodes with photodetector technology and biofeedback sensors integrated with PMT technology. A data processing circuit would be incorporated. The device has laser diodes with photodetector technology and with a biofeedback sensor integrated with PMT technology; as well as in the single layer there is an LED/fiber optic strip with a wavelength range of 250 nm-20,000 nm; data processing circuit with a 3.5-5.0 VDC power supply, CPU, LED/fiber optic connectivity integrated with PMT technology, EEPROM, telemetry, CCD integration, A/D I.O. using USB or other connections with the device having an outer cover away from the skin made of neoprene or other non-allergenic material and a cover next to the skin made of the same material. The unique feature of the single layer device would be that the LED's within the device would themselves have individual wavelengths within the range of 250 nm-20,000 nm and would be enabled to individually turn on and off as a unit as required by therapy.

The Joint/Tissue Inflammation Therapy and Monitoring Device or “JITMon” as shown in FIGS. 10, 15 and 17 is designed to be worn on various parts of the body to treat the area associated with that part of the body. Further, the device is to be made of a neoprene elastic material like that found in wetsuits. Sizing of the device will be accomplished with Velcro type straps allowing for an easy and comfortable fit. Each JITMon will have strategically located Light Emitting Diodes (LED's) with calibrating wavelengths and modulated light frequencies to allow for controlled heat/energy and muscular therapy to an area of inflammation. Additional monitoring devices, using optical fiber, photodetector and photoresin technologies, are integrated into the JITMon device and will be supported with customized software and hardware. This information and technology will allow a physician or therapist to monitor and record vital information such as blood flow in the area, skin temperature and moisture to an external monitoring device.

FIGS. 15, 16 and 17, for example, describe the device as it is to be worn on the body and FIG. 18A and FIG. 18B describe a fiber optic arrangement. Many of the electronic components used in the device are interchangeable and are represented by the following numbered components.

1) Data processing circuit with a 3.5-5.0 VDC power supply, CPU, LED/fiber optic connectivity integrated with PMT technology, EEPROM, telemetry, CCD integration, A/D I.O. using USB connections (80).

2) Laser diodes with photodetector technology and biofeedback sensors integrated with PMT technology (82).

3) LED/fiber optic strips with wavelength control technology range 250 nm-20,000 nm (84).

4) The exterior/interior surface material is neoprene or other non-allergenic material (24 and 26).

FIGS. 15 and 16 describe the device to be worn on the knee. In FIG. 15 the device 90 is shown with a patella cushion ring 92 with LED fiber optics. The blocked off 10 portion 16-16 is shown in detail in FIG. 16.

FIG. 17 is a representation of the JITMon device, with LED therapy being applied to the hand. Sensors attached to the hand (not shown) signal the processor regarding bodily conditions such as heat or moisture so that optimum treatment conditions can be obtained.

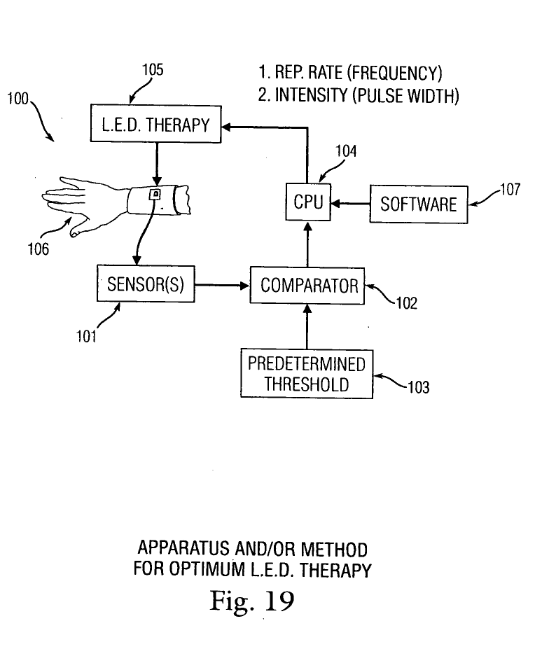

With reference to FIG. 19, an important feature of the present invention involves a feedback loop to optimize the LED therapy, consonant with the multi-tiered (multi-level) LED apparatus and method.

This valuable aspect and feature of the present invention is illustrated, schematically, by the block diagram of FIG. 19.

The feedback loop 100 includes a sensor (or sensors) 101 (as for example, sensing skin temperature) and applying a signal to a comparator circuit 102 for comparison with a predetermined threshold 103. When the signal from the sensor 101 exceeds the predetermined threshold 103, or otherwise detects a change, a central processor unit (“CPU”) 104 adjusts the LED apparatus 105 for the therapy being applied to the patient 106, accordingly. The CPU 104 is suitably programmed by software 107.

As shown schematically in FIG. 19, the frequency or repetition rate (“rep rate”) of the LED apparatus 100, as well as its intensity or pulse width, may be varied for optimum results; and as appreciated by those skilled in the art, the tier (or level) or component of the LED apparatus 100 (hereinbefore described) may also be adjusted and/or applied, selectively, to the patient 106.

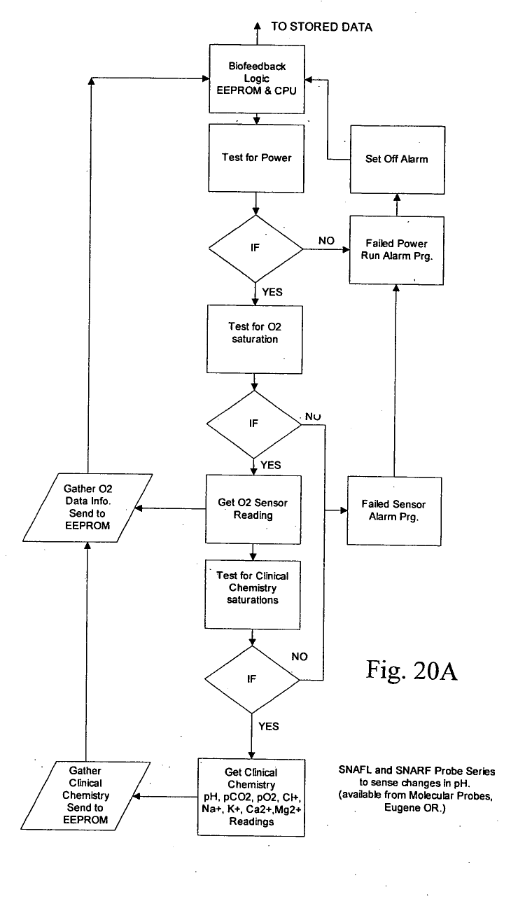

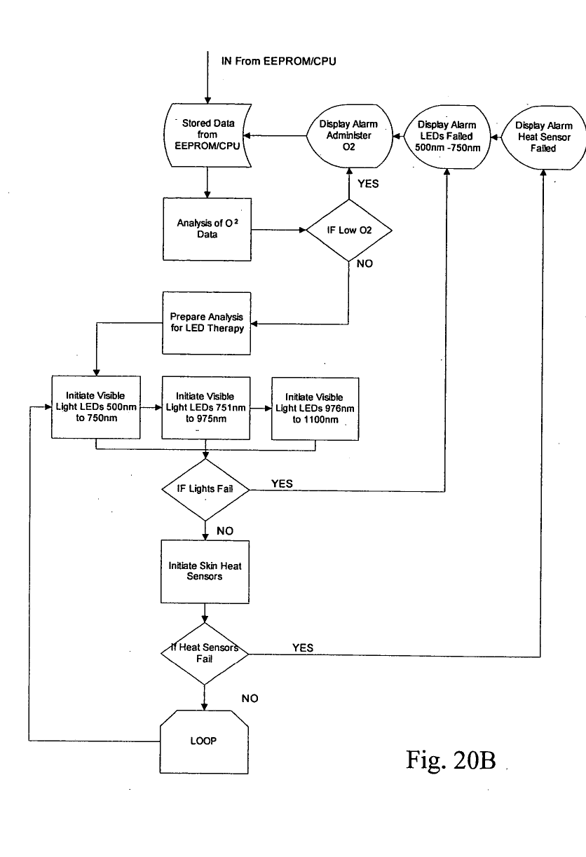

FIGS. 20A and 20B describe a flow-chart involving steps and conditions involving sensors and processor as would be used in the disclosed invention.

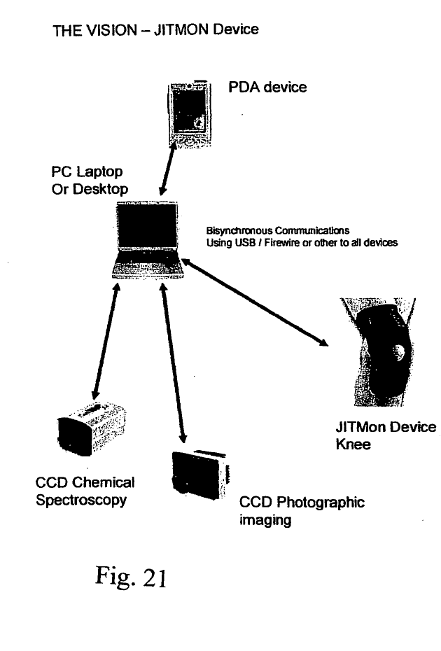

FIG. 21 shows components to be employed with the JITMon Device.

While the invention has been described in terms of inorganic light emitting diodes, the invention will function equally well with organic light emitting diodes (OLED). This type of organic light emitting diode is shown in U.S. Pat. No. 5,955,834. See also Laser Focus World, February 2005. Accordingly, where LED's are mentioned in the claims organic, as well as, inorganic LED's are encompassed.

In carrying out the invention blood flow, skin temperature and moisture are to be monitored to determine modulated light frequencies and wavelength are to be taken into consideration. Exemplary of the manner in which these factors will be taken into consideration are well known to those knowledgeable in sensors of various types.

By surrounding the injured or inflamed areas with an elastic fitted device, which applies controlled heat/energy using Light Emitting Diodes, (LED's) at specific modulated light frequencies and wavelengths will enhance the recovery process. Understanding of the anatomy and physiology of the body and its process to healing, helps the physician or therapist apply proper heat/energy where needed. This therapy improves blood flow and enhances the natural release of cells and chemicals to improve the overall recovery of the patient. Each device will be made to fit the areas of the joints and skeletal system especially the neck, thoracic, knee, elbow, and tarsal and carpal appendages. The device will have the ability to integrate optical fiber and photoresin technology, solid state detectors, sonic/ultrasonic transducers or other high level inputs.

Exemplary of the method for carrying out the invention, the inventors describe the following cases.

Scenario #1

Place your self in a situation where you have twisted your knee by tripping off a sidewalk while crossing to the other side of the street. At first you have mild pain and think little about. You continue your normal routine throughout the day, sitting, standing, walking . . . until you get home late that evening and notice a tremendous amount of swelling around the knee and warmth up your leg.

Thinking about this problem you decide to place Deep Heat© on the knee and then decided to go to bed for the rest of the evening, believing that this would solve the problem before waking the next morning. Upon arising you notice the swelling has subsided but you still have a dull pain in the knee joint. You decide to place an Ace Bandage© over the knee and go to work.

Many weeks pass and you still are having problems with your knee and decide to go to an orthopedic surgeon to get a better evaluation of what has happened. After x-rays and review the doctor tells you the problem was a severe sprain and that at this time you need to wear a knee brace, go to physical therapy for several weeks, take medicine for the edema and pain and possibly lose time at work.

Now at this point, there is a strong need for a device such as the type that the inventors have designed to be applied around the injured site of the knee. Looking at FIGS. 10, 15 and 17, note the shape and general configuration of the device. Here is how it would generally work:
1. A diagnosis is made by the proper physician, showing deep tissue injury.
2. A choice is made by the patient and surgeon on using a noninvasive device for treatment
3. The JITMon Device is selected and placed completely around the injured and inflamed site.
4. A diagnostic reading is taken in real time mode and transferred to a microprocessor within the device and prepared to be sent to an external diagnostic device, such as a laptop or PDA Device.
5. The readings consist of skin temperature, pH, and clinical chemistry (K⁺, Ca²⁺, Mg²⁺, Na⁺ . . . ) blood flow, moisture sensitivity, O₂ saturation and neural sensitivity (See Program Logic Flow Chart).
6. Based on these readings, recommendations via electronic analysis will be made as to the amount, frequency, depth and time period (duration) need to penetrate and irradiate the site. It will also recommend whether the signal should be continuous or pulsed.
7. The physician can use the recommended setting or can manually set the device to levels that can be activated for specific conditions via a laptop or PDA device.
8. The patient then wears the device for the recommended period of time, much like an Ace Bandage. With the induced light and proper wavelengths, the internal edema will begin to dissipate and the natural healing process will be excited allowing for quicker recover and healing of the site.
9. As the patient wears the device a constant circuitry is established to sense skin temperature and determines if the device should be on or off, allowing for controlled heat to the area.
10. Additional circuitry, with fiber optics will allow for the device to be connected with a Charged-Coupled Device (CCD) allowing for an image to be processed of the injured site. This technology is known as Fluorescence Lifetime Imaging (FLIM) and is based on the excitation of protons via light wavelengths and is regarded as a microscopic optical analog of MRI.
   Scenario #2

The same example as Scenario #1 but this time the patient decides not to go to a physician. What can you do? You have placed a dermal ointment (Deep Heat©) on your knee and even applied a knee brace but little or nothing has occurred. Your knee feels better with the support but over several days there is still a problem with swelling and stiffness.

The patient does not have the time to take off or the insurance to cover this type of accident. Another version of the JITMon device now comes into play. This device is similar to the above device but it only has an external manual setting. The patient can acquire this device as an “over-the-counter” sale through a pharmacy or distribution by other means. Once acquiring this device, the patient places the device on the injured site, just like a knee brace and sets the device to “Low”, “Medium”- or “High”. Similar to a heating blanket or heat pad. This allows for a certain amount of light and energy to be focused on the injured site, relieving pressure to the site and stimulating blood flow which in turn reduces the edema. The patient also receives another benefit, the calming warmth they receive as thought they have placed a dermal ointment (Deep Heat© on the site.

In general, the numeric amounts and ranges as used in this patent are intended to be approximate amounts as to be understood by those skilled in the art.

Obviously, many modifications may be made without departing from the basic spirit of the present invention. Accordingly, it will be appreciated by those skilled in the art that within the scope of the appended claims, the invention may be practiced other than has been specifically described herein.

1. A phototherapy device for light and heat treatment and for reducing inflammation, edema and/or medical conditions associated with the joints, muscles, wound healing, nerves and skin tissue of a human or animal subject comprising, a portable device configured to be worn in contact with the skin and surrounding the area or areas of pain, inflammation, edema, neural and/or muscular damage with the device being configured to have multiple layers of LED's having varying wavelengths with the multiple layers of LED's being distributed in a range of near contact with the skin tissue to a few centimeters above the skin tissue and also having a power supply for the LED's and a switch for on/off control.

2. The phototherapy device of claim 1 wherein multiple different LED's each emitting light of a discrete range of wavelength and in combination cover the entire spectrum from ultraviolet to near infrared.

3. The phototherapy device of claim 1 is configured with multiple layers of LED's having wavelengths in the range of 250 nm to 20,000 nm.

4. The phototherapy device of claim 3 configured with three layers wherein the LED layer closest to the skin has wavelengths in the range of 250 nm-500 nm; the next level has wavelengths in the range of 500-700 nm and that furthest from the skin has wavelengths in the range of 700-20,000 nm.

5. The phototherapy device of claim 1 is configured with four layers of LED's having wavelengths in the range of 250 nm to 20,000 nm.

6. The phototherapy device of claim 5 wherein the LED layer closest to the skin has wavelengths in the range of 250 nm-500 nm, the second level has wavelengths in the range of 500-700 nm, the third level has wavelengths in the range of 700-900 nm and the fourth level has wavelengths in the range of 700-20,000 nm.

7. The phototherapy device of claim 1 wherein the LED's working mode is a CW light mode, modulated light mode, or pulsed light mode.

8. The phototherapy device of claim 1 wherein the device is configured with multiple LED's having light wavelengths in the range of 250 nm to 20,000 nm and modulated or pulsed light frequencies of 1 Hz through 1 GHz.

9. The phototherapy device of claim 1 being further provided with integrated low voltage power, electronic memory and communications via analog/digital connection or telemetric medical sensor; allowing for independent control of tissue temperature and operating and resting (“rest mode”) device working mode, modulation of the light frequencies, intensities and wavelengths of the LED's.

10. The phototherapy device of claim 1 being provided with multiple types of CW, pulsed or modulated light intensities of LED's of multiple spectral ranges and fiber optics to deliver light to the areas associated with edema, inflammation, neurological and muscular skeletal regions and to read out physiological and biochemical changes associated with edema, inflammation, neurological and muscular skeletal regions.

11. The phototherapy device of claim 1 being provided with multiple types of CW, pulsed or modulated light intensities of Laser Diode Technology of multiple spectral ranges and fiber optics to deliver light to the areas associated with edema, inflammation neurological and muscular skeletal regions and to read out physiological and biochemical changes associated with edema, inflammation, neurological and muscular skeletal regions.

12. The phototherapy device of claim 1 being provided with multiple types of CW, pulsed or modulated light intensities of LED's including Laser Diode Technology and fiber optics to deliver light to the area associated with edema, inflammation, neurological and muscular skeletal regions and to read out physiological and biochemical changes within the area or areas of inflammation, edema, neurological and muscular skeletal regions.

13. The phototherapy device of claim 1 being provided with software to be integrated into a personal computer or hand held device allowing for the monitoring and documentation of information accumulated, identifying wavelengths, light intensities, light modulated frequencies, localized heat and heat variances, physiological and biochemical changes, skin temperature and other biometrics as needed associated with the subject as it applies to the localization of the device.

14. The phototherapy device of claim 1 being provided with the integration of laser and/or laser diodes and photodetector technology allowing for data to be gathered, stored and retrieved in both “real time” and historical events coupled with PMT technology, Spectral Technology and CCD technology.

15. The phototherapy device of claim 1 wherein the multiple layers of LED's and fiber optics have multiple types of modulated light intensities of LED's of multiple spectral ranges and fiber optics distributed in a range of near contact with the skin to a few centimeters from the skin tissue; with orientation toward the subject.

16. The phototherapy device of claim 1 being further provided with fiber optics.

17. The phototherapy device of claim 1 being further provided with multiple layer technology integrating the light emitting diodes (LED's), laser diodes, fiber optic strands fiber optic bundles and polarization light optics.

18. The phototherapy device of claim 1 constructed of a neoprene type material or other non-allergenic material(s), allowing the elasticity, permeability, flexibility, protection and comfort to the injured site of the patient.

19. The phototherapy device of claim 1 designed to accommodate multiple pediatric and adult sizes of small, medium, large and extra-large and with wrap-around Velcro® type adhesive/connectivity or other connective material allowing for a secure fit.

20. The phototherapy device of claim 1 use for cosmetic purposes with multiple construction designs of elastic portable device for different body parts.

21. The phototherapy device of claim 1 being further provided with Light Guide Technology for uniform illumination of the skin and surrounding the area or areas of inflammation, edema, neural and/or muscular damage over short and long periods of time.

22. The phototherapy device of claim 1 where in the device worn in contact with the skin is elastic.

23. A method comprising applying to the skin of a patient phototherapy a device configured with multiple layers of LED's having varying wavelengths and modulated/pulsed light frequencies and fiber optics such that the patient or a healthcare professional will be able to choose a therapeutic treatment from one or all of the following ranges,
a) a first range of 250 nm to 500 nm to penetrate the dermal and sub-dermal tissue allowing for a stimulation of blood cells, vitamins, proteins, molecular genetic material, amino acids, enzymes and other physiological mechanics to be implemented for the reduction of edema and discoloration to the injured site,
b) a second range of light, of 500 nm to 700 nm for deeper penetration of the tissue into the structure of the muscles, fibers and tendons, down to the skeletal structure for stimulating the natural process of healing by invoking physiological processes to improve the reduction of edema, stimulation of blood circulation, reduction of neural edema, regeneration of tissue and improved enzymatic processes, to the injured site,
c) a third range or layer of light, of 700 nm to 20,000 nm allowing for the penetration of tissue, down to the skeletal structure for stimulating the natural process of healing by invoking physiological processes to improve the reduction of edema, stimulation of blood circulation, reduction of neural edema, regeneration of tissue and improved enzymatic processes, and inducing heat and deep tissue therapy,
d) diagnostic therapy which will be transitive to polarized or non-polarized light.

24. A method comprising applying to the skin of a patient phototherapy a device configured with multiple layers of LED's having varying wavelengths and modulated/pulsed light frequencies and fiber optics such that the patient or a healthcare professional will be able to choose a therapeutic treatment from one or all of the following ranges,
a) a first range of 250 nm to 500 nm to penetrate the dermal and sub-dermal tissue allowing for a stimulation of blood cells, vitamins, proteins, molecular genetic material, amino acids, enzymes and other physiological mechanics to be implemented for the reduction of edema and discoloration to the injured site,
b) a second range of light, of 500 nm to 700 nm for deeper penetration of the tissue into the structure of the muscles, fibers and tendons stimulating blood cells, vitamins, proteins, molecular genetic material, amino acids, enzymes and other physiological mechanics to be implemented for the reduction of edema and discoloration to the injured site,
c) a third range of light, of 700 nm 900 nm allowing for the penetration of tissue, down to the skeletal structure for stimulating the natural process of healing by invoking physiological processes to improve the reduction of edema, stimulation of blood circulation, reduction of neural edema, regeneration of tissue and improved enzymatic processes,
d) a fourth layer of light furthest from the skin in the range of 900 to 20,000 nm, providing additional therapeutic care by inducing heat and deep tissue therapy and
e) diagnostic therapy which will be transitive to polarized or non-polarized light.

25. The phototherapy device of claim 1 where in a non-light related heat generated from the device electronics including LED's, laser diodes is used as a part of phototherapy.

26. The phototherapy device of claim 1 being provided with a thin film lightly transparent colored blue to allow light diffusion to occur.

27. The phototherapy device of claim 1 wherein the tiers of LED's are individually programmed to turn “on” or turn “off” as the therapeutic needs require.

28. The phototherapy device for light and heat treatment of claim 1 wherein the device is provided with at least one sensor for monitoring a condition of a patient as light therapy is applied to the patient and a feedback loop responsive to the sensor for adjusting the light source of the multiple layers of LED's thus optimizing the light therapy applied to the patient.

29. A method for treating a patient in need of phototherapy comprising applying the phototherapy device of claim 9 to a patient while electronically monitoring a physiological state in said patient and remotely electronically altering a treatment parameter in the phototherapy device based on said monitoring of said physiological state.

30. A phototherapy device for light and heat treatment and for reducing inflammation, edema and/or medical conditions associated with the joints, muscles, wound healing, nerves and skin tissue of a human or animal subject comprising, a portable device configured to be worn in contact with the skin and surrounding the areas of pain, inflammation, edema, neural and/or muscular damage with the device being configured to have a polarized film or light guide which is to be applied to the skin of a body part, said polarized film or light guide accepting white light and transmitting a light through said polarized film or light guide and with the treatment conditions of the patient being electronically sensed and light being applied in response to the electronic sensing.

31. The phototherapy device of claim 30 wherein the polarized film or light guide is replaceable and is held in place by a clip.

32. The phototherapy device of claim 30 wherein the light enters the polarized film or light guide longitudinally at the end of the polarized film or light guide.

33. In an apparatus for diagnosing and/or treating a patient's medical problem with light therapy, the improvement which comprises a multi-tiered light source, at least one sensor for monitoring a condition of the patient as the light therapy is applied to the patient, and a feedback loop responsive to the sensor for adjusting the multi-tiered light source for optimizing the light therapy applied to the patient.

34. The improvement of claim 33, therein the multi-tiered light source comprises a plurality of LED's.

35. The improvement of claim 34, wherein the frequency of the plurality of LED's is adjusted by the feedback loop.

36. The improvement of claim 33, wherein the apparatus comprises a flexible bandage.

37. The improvement of claim 33, therein the multi-tiered light source has different frequencies.

38. The improvement of claim 33, wherein the sensor monitors the temperature of the patient's skin tissue.

39. The improvement of claim 33, wherein the sensor monitors oxygen levels of the patient's skin tissue.

40. The improvement of claim 33, wherein the feedback loop adjusts the pulse width and/or the rep rate of the light therapy.
